(19) United States
(12) Patent Application Publication
(10) Pub. No.: US 2005/0177092 A1
(43) Pub. Date: Aug. 11, 2005

(54) Apparatus and assembly for administering antimicrobial agent

(76) Inventors: Hirotoshi Adachi, Ibaraki (JP); Naruhito Higo, Ibaraki (JP); Shuji Sato, Kanagawa (JP); Aarti Naik, Archamps (FR); Yogeshvar N. Kalia, Archamps (FR); Richard H. Guy, Archamps (FR)

Correspondence Address: TOWNSEND & BANTA;c/o PORTFOLIO IP, PO BOX 52050, MINNEAPOLIS, MN, 55402, US

(21) Appl. No.: 10/512,442

(22) PCT Filed: Apr. 25, 2002
(86) PCT No.: PCT/JP02/04147

(51) Int. Cl. A61N 1/30

(52) U.S. Cl. 604/20

(57) ABSTRACT

A device and assembly for administering antimicrobial for killing resident microorganisms on human skin and thereby effectively preventing infections caused by the microorganisms, are provided.

The device for administering antimicrobial of the invention uses iontophoresis, and it comprises: a donor electrode (100) containing an antimicrobial; a reference electrode (110) provided as a counter electrode of the donor electrode; and a power unit (120) for applying current between the donor electrode and the reference electrode in such a manner as to allow the total amount of current applied to be 1 to 30 mA·min/cm². The area on the skin to which the donor electrode (100) is applied is suitably 1 to 100 cm². Thus, the catheter or injection needle insertion site on human skin is disinfected.

### TECHNICAL FIELD

The present invention relates to a device for administering antimicrobial for example, killing resident microorganisms on the skin at the catheter or injection needle insertion site and an assembly thereof, in particular, to a device for administering antimicrobial for delivering an antimicrobial to human skin effectively and safely on the basis of active absorption control and an assemblage thereof.

### BACKGROUND ART

Catheter indwelling, which is frequently done in basic and daily medical service for inpatient management, is likely to induce a variety of complications, ranging from fever to septicemia, if the catheter is used inadequately. To contrive measures to prevent intravenous catheter-related infections has been one of the subjects of infection prevention study for many years. The intravenous catheter-related infections have been a global issue because of their rapid increase in current medical care and have imposed a problem of high medical service fee and hospital costs on people [Mermel L. A., Prevention of intravascular catheter-related infections, Infect. Dis. Clin. Parc., 1994, 3, 391-398]. Above all, the severest infections have been observed in central venous catheter indwelling, and even in recent years in which patient management in intensive care units has made progress, the incidence of intravenous catheter-related infections and the mortality rate of patients having such infections have been high [Mermel L. A., Prevention of intravascular catheter-related infections, Ann Intern Med., 2000, 132, 391-402].

In indwelling of catheter, which is a foreign matter to living bodies, in a vein, fibrin adheres to the catheter at the time of puncturing the vein. And starting from this, a coat of fibrin or thrombus adheres to the surface of the catheter. It is said that the catheter with a coat of fibrin or thrombus adhering to its surface becomes a nest of bacterium growth and thereby causes catheter-related infections. The bacteria isolated most commonly in catheter-related infections include, for example, coagulase-negative staphylococci (mostly Staphylococcus epidermidis) and Staphylococcus aureus [Das I., Philpott C., and Gerorge R. H., Central venous catheter-related septicemia in pediatric cancer patients, J. Hosp. Infect., 1997, 36, 67-76., Gupta B., Bernardini J., and Piraino B., Peritonitis associated with exit site and tunnel infections, Am. J. Kidney Dis., 1996, 28, 415-419].

Bacterium invasion pathways include, for example, contamination through a catheter connection portion, contamination of injection solutions, contamination through the skin of catheter insertion site and contamination at the time of catheter puncture. Above all, contamination through a catheter connection portion, contamination through the skin of catheter insertion site and contamination at the time of catheter puncture are considered to be primary causes.

On the other hand, improvement of catheters themselves has been marked and rapid. Specifically, in respect to contamination through the skin of catheter insertion site, ascending infections from a catheter insertion site via the outer surface of a catheter can be prevented by making a tunnel under skin from the catheter insertion site, making a catheter exit at some other site, and arranging a Dacron cuff at the catheter exit to accelerate the growth of subcutaneous tissue [Broviac J. W., Cole J. J. and Scribner B. H., A silicon rubber atrial catheter for prolonged parenteral alimentation, Surg. Gynecol Obstet., 1973, 136, 602-606., Hickman R. O., Buckner C. D., Clift R. A. et al, A modified right arterial catheter for access to the venous system in marrow transplant recipients].

Furthermore, various attempts, such as polymer modification in which polymer is impregnated with disinfectant or antibody, have been made so as to inhibit the growth of bacteria on catheter walls [Maki D. G., Cobb L., Garman J. K., et al., An attachable silver-impregnated cuff for prevention of infection with central venous catheters. A prospective randomized multi-center trial, Am. J. Med., 1988, 85, 307-314., Heard S. O., Wagle M., Vijayakumar E., et al., Influence of triple-lumen central venous catheters coated with chlorhexidine and silver sulfadiazine on the incidence of catheter-related infections, Arch. Intern. Med., 1998, 158, 81-87., Tennenberg S., Lieser M., McCurdy B., et al., A prospective randomized trial of an antibiotic- antiseptic-coated central venous catheter in the prevention of catheter-related infections, Arch. Surg., 1997, 132, 1348-1351]. The methods of these prior arts are effective in catheter-related infections and have caused a remarkable improvement in catheters.

In recent years, induction of infections at the time of catheter insertion by resident bacteria on human skin has received great attention as a cause of catheter-related infections such as septicemia. In particular, bacteria such as staphylococci, which are known to induce serious infections, are widely distributed on the surface of human skin and are concentrated on the stratum corneum or the appendages such as sebaceous gland [Eady E. A., Sampling the bacteria of the skin, in: Serup J., and Jemec G. B. E., eds., Handbook of non invasive methods and the skin, Boca Raton, CRC ress, 1995., Kearney J. N., Harnby D., Gowland G., and Holland K. T., The follicular distribution and abundance of resident bacteria on human skin, J. Gen. Microbio., 1984, 130, 797-801., Selwyn S., and Ellis H., Skin bacteria and skin disinfection reconsidered, Br. Med. J., 1972, 1, 136-140].

As a method of disinfecting human skin to kill these bacteria, disinfectants such as povidone-iodine have been used; however, it is reported that such disinfectants cannot kill the bacteria perfectly [Hendley J. O., and Ashe K. M., Effect of topical antimicrobial treatment on aerobic bacteria in the stratum corneum of human skin, Antimicrob Agents Chemother., 1991, 35, 627-631]. The conventional disinfection methods using disinfectants cannot produce sufficient disinfection effect particularly on resident bacteria on human skin which inhabit the appendages such as pore, sebaceous gland and sweat gland. This is possibly because lipids covering the stratum corneum surface or the appendages limit the permeation of drugs and retard the movement of active drugs to the site which resident bacteria on human skin inhabit [Price P. B., The bacteriology of normal skin; a new quantitative test applied to a study of the bacterial flora and the disinfection action of mechanical cleansing, J. Infect. Dis., 1939, 63, 301-318., Selwyn S., and Ellis H., Skin bacteria and skin disinfection reconsidered, Br. Med. J., 1972, 1, 136-140., Sato S., Sakuragi T., and Dan K., Human skin flora as a potential source of epidural abscess, Anesthesiology, 1996, 85, 1276-1282].

Skin is a major barrier against various substances and limits the percutaneous permeation of highly polar or charged drugs quite frequently. Furthermore, the total area of the openings of the skin appendages such as pore, sebaceous gland and sweat gland, which are known as shunt pathways, is small and the lipid-soluble substances discharged from the appendages also limit the percutaneous absorption of highly polar or charged drugs [Potts R. O., and Francoeur M. L., the influence of stratum corneum morphology on water permeability, J. Invest. Dermatol., 1991, 96, 495-499., Barry B. W., Dermatological formulations: Percutaneous absorption, In: Swarbrick J. ed., Drugs and the pharmaceutical Sciences, New York and Basel, Marcel Dekker Inc., 1983].

Iontophoresis is a percutaneous absorption accelerating system using electricity as an external stimulation, and the principle upon which it accelerates the permeation of molecules into the skin barrier is that an electric field produced between an anode and a cathode by applying current across them causes the movement of positively charged molecules from the anode to the cathode and negatively charged molecules from the cathode to the anode (electric repulsion) as well as the movement of water from the anode to the cathode (electroosmosis).

In other words, iontophoresis is a method of allowing charged drugs to be actively absorbed into the body by electrochemical potential. It is, for example, a method of administering positively charged drugs to the skin from the anode side. As to the polar drugs, it is known that since current concentrates on appendages in the stratum corneum, such as sweat gland and hair follicle, of which electrical resistance is low, they are absorbed into the skin through such appendages, as pathways. On the other hand, for the substances which are not charged, they are allowed to move with the movement of water, as a solvent, when producing an electric field between an anode and a cathode. Thus, in iontophoresis, not only passive diffusion but also electric repulsion and electroosmosis play an important part in drug absorption [Banga A. K. ed., Electrically assisted transdermal and topical drug delivery, London and Bristol, Taylor & Francis Ltd., 1998., Merino V., Alberti I., Kalia Y. N., et al, Transdermal and skin-targeted drug delivery, J. Cut. Med. Surg., 1998, 2, 108-119., Merino V., Kalia Y. N., and Guy R. H., Transdermal therapy and diagnosis by iontophoresis, Trends Biotechnol., 1997, 15, 288-290., Turner N. G., and Guy R. H., Iontophoretic transport pathways: Dependence on penetrant physicochemical properties, J. Pharm. Sci., 1997, 86, 1385-1389].

Accordingly, iontophoresis is an optimum method of delivering drugs topically to the targeted site such as skin appendages; in addition, since it is noninvasive, much hope can be placed on it as a reliable and convenient method. However, there have been no detailed reports on apparatus for delivering antimicrobials against resident bacteria on the skin, in other words, the apparatus for delivering active ingredients having antimicrobial activity specifically to the appendages.

U.S. Pat. No. 5908401 specification discloses an iontophoresis apparatus which is suitable for percutaneous administration of antivirals. The patent, however, neither describes suitable delivery of antivirals nor discloses a clinically effective and safe delivering apparatus. Amini T. et al. reports on the effect of iontophoresis on increasing the permeation of chloehexidine gluconate into skin in Journal of Pharmacy and Pharmacology (52 (Supplement), p.25, 2000). This report indicates that the permeation of drugs into the skin varies with pH of solution. There is, however, no examination on antimicrobial activity, moreover, no description of clinically effective iontophoresis apparatus in this report. Thus, the optimum drug administering apparatus is still unknown.

Accordingly, the object of the invention is to provide an device for administering antimicrobial for killing resident microorganisms on human skin and thereby effectively preventing infections caused by the microorganisms and an assemblage thereof.

### DISCLOSURE OF THE INVENTION

The inventors of the invention concentrated their energy on examining the solution of the problem attendant to the apparatus currently in use, that is, the problem of not having the capability of producing sufficient disinfection effect. After the intensive examination, the inventors came up with an apparatus that can be used specifically for the skin appendages, which microorganisms inducing catheter-related infections inhabit, quickly and efficiently and is not only excellent in, for example, skin stimulation safety but easy and safe to use, and they finally made the invention. Specifically, the invention is an apparatus which contains an antimicrobial and adopts iontophoresis in which the total amount of current applied is 1 to 30 mA·min/cm², the apparatus enabling active ingredients to be delivered directionally to the skin appendages in a reliable manner and in a short period of time and the antimicrobial to exhibit its activity to living bodies safely and efficiently.

Furthermore, the inventors came up with an assemblage which enables antimicrobial effect to be significantly enhanced and disinfection to be carried out in a shorter period of time and in a more effective manner, after additional intensive examinations, and finally made the invention. Specifically, the invention is an assemblage which is a combination of a passive apparatus and an active apparatus and, for example, an alcohol preparation can be applied to the passive apparatus and an iontophoresis apparatus can be applied to the active apparatus. If the apparatus or assemblage of the invention is combined with a local analgesic, patients' pain at the time of catheter punctuation can be gotten rid of. This may increase patient compliance at the same time.

Specifically, the invention is
(1) an device for administering antimicrobial adopting iontophoresis for disinfecting the skin at the catheter or needle insertion site, the apparatus including:
   (a) a donor electrode containing an antimicrobial;
   (b) a reference electrode provided as a counter electrode of the donor electrode; and
   (c) a power unit for passing current between the donor electrode and the reference electrode in such a manner as to allow the total amount of current applied to be 1 to 30 mA·min/cm².
(2) The area on the skin to which the donor electrode is applied is suitably 1 to 100 cm².
(3) The antimicrobial is at least one selected from the group consisting of, for example, antiviral, antibacterial, chemotherapeutic, antibiotic, disinfectant and antimycotic.
(4) The antimicrobial has antimicrobial activity against at least one selected from the group consisting of, for example, Staphylococcus aureus, Staphylococcus epidermidis, coagulase negative staphylococci, Micrococci, gram-positive bacilli, gram-negative bacillus acinetobacter, glucose-nonfermenting gram-negative rods, Candida, Serratia and methicillin-resistant bacteria.
(5) The donor electrode is anodic and the antimicrobial contained therein may be chlorhexidine or the salts thereof.
(6) The donor electrode may further contain a local analgesic.
(7) The donor electrode is anodic and the local analgesic contained therein may be lidocaine or the salts thereof.
(8) The donor electrode may further contain a vasoconstrictor.
(9) The donor electrode is anodic and the vasoconstrictor contained therein may be epinephrine or the salts thereof.

Further, the invention is
(10) an antimicrobial administering assemblage for disinfecting the skin at the catheter or needle insertion site, the apparatus including:
   (a) a first component including a passive apparatus for disinfecting the skin surface over a wide range; and
   (b) a second component including an active apparatus for disinfecting topically the skin surrounding the catheter or needle insertion site.
(11) Each of the first and second components may contain an antimicrobial.
(12) A first area as an area on the skin to which the first component is applied is suitably wider than a second area as an area on the skin to which the second component is applied and the first area is 20 cm² or more and the second area is 1 to 100 cm².
(13) The first component may contain alcohols.
(14) The alcohols may be ethanol or isopropyl alcohol.
(15) The second component may include:
   (a) a donor electrode containing an antimicrobial;
   (b) a reference electrode provided as a counter electrode of the donor electrode; and
   (c) a power unit for passing current between the donor electrode and the reference electrode in such a manner as to allow the total amount of current applied to be 1 to 30 mA·min/cm².
(16) The antimicrobial contained in the first or second component is at least one selected from the group consisting of, for example, antiviral, antibacterial, chemotherapeutic, antibiotic, disinfectant and antimycotic.
(17) The antimicrobial contained in the first or second component has antimicrobial activity against at least one selected from the group consisting of, for example, Staphylococcus aureus, Staphylococcus epidermidis, coagulase-negative staphylococci, Micrococci, gram-positive bacilli, gram-negative bacillus acinetobacter, glucose-nonfermenting gram-negative rods, Candida, Serratia and methicillin-resistant bacteria.
(18) The first and the second components may contain the same antimicrobial.
(19) The donor electrode of the second component is anodic and the antimicrobial contained therein may be chlorhexidine or the salts thereof.
(20) The donor electrode of the second component may further contain a local analgesic.
(21) The donor electrode of the second component is anodic and the local analgesic contained therein may be lidocaine or the salts thereof.
(22) The donor electrode of the second component may further contain a vasoconstrictor.
(23) The donor electrode of the second component is anodic and the vasoconstrictor contained therein may be epinephrine or the salts thereof.

The device for administering antimicrobial thus constructed is capable of inhibiting the incidence of infections caused by resident microorganisms on human skin and may contribute largely to the field of health care by holding down lethality associated with the incidence of catheter-related septicemia and reducing the health care costs associated with the treatment.

### BRIEF DESCRIPTION OF THE DRAWINGS

FIG. 1 is a view of one example of the device for administering antimicrobial constructed in accordance with the invention.

FIG. 2 is a view of one example of the assembly for administering antimicrobial constructed in accordance with the invention.

FIGS. 3 are graphs showing the influence of drug concentration on the activity of skin bacteria in iontophoresis, FIG. 3(a) is a graph for a passive administration group and FIG. 3(b) a graph for an iontophoretic administration group.

FIG. 4 is a graph showing the influence of the total amount of current applied on the activity of skin bacteria in iontophoresis.

FIG. 5 is a graph showing the influence of antimicrobial administering methods on the activity of skin bacteria.

### BEST MODE FOR CARRYING OUT THE INVENTION

The invention will be described in detail with reference to the accompanying drawings as the need arises, hereinafter.

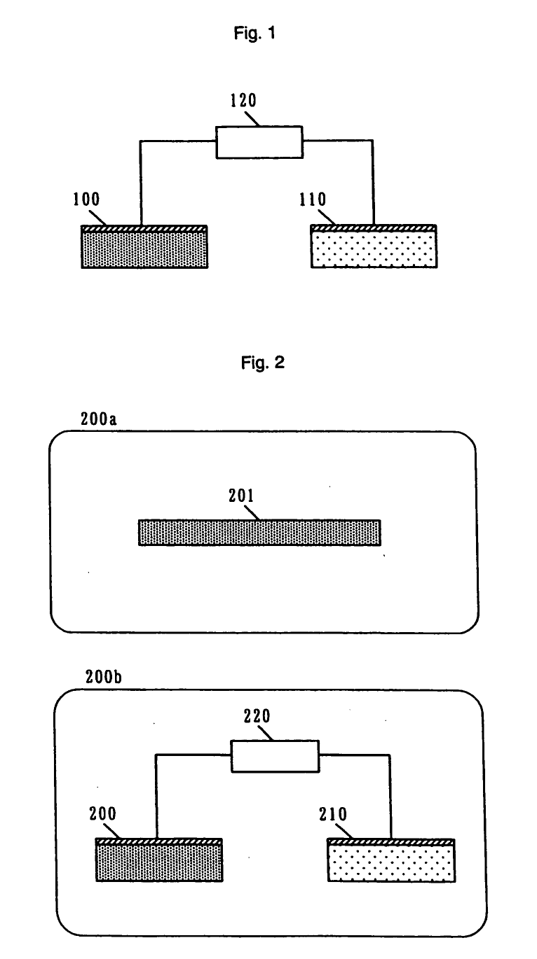

Referring to FIG. 1, there is shown a view of one example of device for administering antimicrobial constructed in accordance with the invention. As shown in the figure, this apparatus includes: a donor electrode 100 containing an antimicrobial; a reference electrode 110 provided as a counter electrode of the donor electrode; and a power unit 120 for passing current between the donor electrode and the reference electrode.

The antimicrobials used as an active ingredient in the invention are preferably compounds having antimicrobial activity. The compounds having antimicrobial activity may be free compounds or the salts thereof; however, the hydrochlorides are particularly preferable.

The antimicrobials include, for example, antivirals, antibacterials, chemotherapeutics, antibiotics, disinfectants and antimycotics. In particular, the antimicrobials include compounds having antimicrobial activity against at least one selected from the group consisting of Staphylococcus aureus, Staphylococcus epidermidis, coagulase-negative staphylococci, Micrococci, gram-positive bacilli, gram-negative bacillus acinetobacter, glucose-nonfermenting gram-negative rods, Candida, Serratia and methicillin-resistant bacteria.

Usually, in device for administering antimicrobial embodying the invention, the donor electrode contains an active ingredient, whereas the reference electrode does not; however, each of the donor and the reference electrodes can contain the active ingredients described above. In another embodiment of the invention, for example, the reference electrode can contain the same active ingredient as the donor electrode does.

As an antiviral, antibacterial or chemotherapeutic are used, for example, acyclovir, vidarabine, saquinavir, lamivudine, valacyclovir hydrochloride, zanamivir, orsetamivir phosphate, norfloxacin, ciprofloxacin, delavirdine mesylate, lopinavir, ritonavir, ofloxacin, levofloxacin, linezolid, teicoplanin, gatifloxacin, pazufloxacinmesylate, prulifloxacin, sitafloxacin hydrate, chymosifloxacin, enoxacin, lomefloxacin hydrochloride, ganciclovir, delavirdine mesylate and lamivudine.

As an antibiotic are used, for example, azithromycin hydrate, gentamicin sulfate, lipidomicin, sisomicin sulfate, tetracycline hydrochloride, ampicillin, cefaclor, cefalexin, cefalotin sodium, cefotiam hydrochloride, cefazolin sodium, thienamycin, sulfazecin, streptomycin sulfate, kanamycin sulfate, rifampicin, vancomycin hydrochloride, lincomycin hydrochloride, fosfomycin, minocycline hydrochloride, rifampicin, clindamycin, amikacin sulfate, ofloxacin, cephocelis sulfate, amoxicillin, clarithromycin, terythromycin, cefamezin sodium, meropenem, biapenem, doripenem and lithipenem apoxyl.

As adisinfectant are used, forexample, benzalkoniumchloride, benzethonium chloride, glutaral and chlorhexidine gluconate, and as an antimycotic are used, for example, amphotericin B, itraconazole, fluconazole, miconazole, micafungin, polyconazole and griseofulvin.

In the invention, one or more appropriately selected from among the above antimicrobials can be used.

In the donor and reference electrodes used in the device for administering antimicrobial adopting iontophoresis in accordance with the invention, their structures and constituent materials are not limited to any specific ones; and the structures include: for example, (1) a matrix type in which an antimicrobial as an active ingredient is impregnated into the electrode or dispersed in hydrogel etc. in the solution state; (2) a reservoir type in which a semi-permeable membrane for holding a conductive layer between a conductive layer and the skin, a selectively permeable membrane for controlling the movement of substances, a regulation membrane for regulating the drug permeation rate, etc. are provided; (3) a stacking type in which a drug holding layer is provided at the time of use so as to enable the application of a high concentration of active ingredient to the skin contact surface (the stacking type structure is useful particularly when the antimicrobial used is chemically unstable or when the drug used exhibits potent pharmacologic effect or is expensive, and this type is used in such a manner as to allow the antimicrobial holding means to be in contact with the hydrophilic conductive layer immediately before use). The shape of electrodes, the state of conductive layers and the distribution state of antimicrobial are not particularly limited and the way of reinforcing electrodes, that is, the form of backing and the arrangement of adhesive layers are not particularly limited, either. Desirably the donor electrode is capable of fully disinfecting the catheter insertion range; however, the excess disinfection over a wide range using the iontophoresis apparatus of the invention should be avoided for safety reasons, and the area on the skin to which the donor electrode is applied is preferably about 1 to 100 cm², more preferably 2 to 50 cm².

Electrolyte, pH adjustor, buffer, skin protector, emollients, stabilizer, thickener, wetting agent, surfactant, solubilizer, dissolution aid, humectnt, absorption accelerator, self-adhesive, tackifier, preservative, etc. may be added to the matrix, reservoir, conductive layer, drug holding layer used in the invention, as long as their performance is not affected.

The electrode materials used in the invention are not limited to any specific ones, provided that they are conductive electrode materials which can be used in the ordinary iontophoresis apparatus. The conductive materials include: for example, silver; silver chloride; aluminum; zinc; copper and iron; as active electrode materials, and carbon; platinum; titanium and stainless steel, as inactive electrode materials. Among all these materials, silver or silver/silver chloride as an active electrode material has excellent electrical properties such as resistance value, and the use of the material in the paste state can provide electrodes at low cost and at high productivity rate. As for the inactive electrode material, the use of carbon, etc can provide electrodes at low cost. These materials can be used in combination with each other.

In the power unit used in the invention, one-way energization is possible in which current is allowed to flow in a fixed direction; alternatively, switching energization is also possible in which the polarity is switched while allowing current to flow. The same power unit as used for the donor electrode can also be used for the reference electrode. When using active ingredients in both electrodes, switching energization is effective means. Multipolar output energization is also possible in which multiple electrodes are arranged. The current output from the power unit is preferably DC iontophoresis from the viewpoint of drug delivering properties, and usually it can be controlled at constant current or constant voltage; however, to control drug absorption closely, constant current control is preferable. The term “current” herein used means transmission current related to drug absorption. In the DC iontophoresis of the invention, direct current, pulse direct current or pulse out-of-polarization direct current can be used. As a power source, one capable of applying continuous DC voltage or pulse DC voltage is preferable. The combination of the above two is also applicable. In pulse direct current, rectangular pulse direct current is applied and the frequency of the pulse DC voltage is appropriately selected from the range of preferably 0.1 to 200 kHz, more preferably 1 to 100 kHz, and particularly preferably 5 to 80 kHz. The on/off ratio of pulse DC voltage is appropriately selected from the range of 1/100 to 20/1, preferably 1/50 to 15/1, and more preferably 1/30 to 10/1. In this energizing means, if voltage to be applied is changed immediately after starting and completing energization, a stimulated sensation on skin can subside.

The optimum current density range of the DC iontophoresis apparatus of the invention is restricted by the skin permeability to antimicrobials or the skin stimulating properties when current is applied. The current density is preferably 0.01 mA/cm² to 1.0 mA/cm² and more preferably 0.05 mA/cm² to 0.5 mA/cm², and the total amount of current to be applied is 1 to 100 mA·min/cm², preferably 1 to 30 mA·min/cm², and more preferably 2 to 15 mA·min/cm². The preferable concentration of the antimicrobial applied as an active ingredient in the invention is not particularly restricted; however, concentrations are preferable around which the skin permeability to the drug or the effect of the drug is not drug-concentration dependent. In that case, concentrations around which the skin permeability to drug, the movement of drug on skin or the pharmacologic effect of drug is not correlated with the total amount of current applied are selected through an in vitro skin permeability test or a therapeutic activity test. Specifically, in ordinary preparations, the concentration is in the range of 0.0001 w/v % to 10 w/v %, preferably 0.001 w/v % to 5 w/v %, and more preferably 0.01 w/v % to 5.0 w/v %, though the drug concentration is influenced by the presence of competitive ion component charged equally to the drug in the donor solution or by the macromolecules such as hydrogel which inhibit drug movement. The reason for this is that in many drugs, active ingredients having antimicrobial activity are charged positive, in addition, the molecular weight (normally around several hundred dalton) related to the drug movement at the time of current application and the physicochemical properties such as lipid-solubility are relatively common among many active ingredients.

Since the transportation efficiency of drugs in iontophoresis is influenced by the electrolyte coexisting with active ingredients, the addition of salts necessary for electrode reaction should be kept to a minimum. For example, when the active ingredients are charged positive and the electrodes consist of active electrode materials such as silver, as a method of minimizing the influence of competitive ions, (1) hydrochlorides of antimicrobial are used, (2) hydrochlorides of resin or macromolecule (e.g. anion exchange resin (Cholestylamine) or amino alkyl methacrylate copolymer E (Oydragid E100, Oydragid EPO, Plastoyd E35L)) are used, and (3) a minimum concentration of chlorine ion P (mmol) calculated from the following equation (I)
P=I×T/96500·n (I)
wherein I and T represent energizing duration (second) and average transmission current (mA), respectively, and the transport number of chlorine ions (normally 0.6) is used.

Another apparatus embodying the invention is an apparatus which delivers an antimicrobial and a local analgesic simultaneously by iontophoresis so as to get rid of patients' pain at the time of catheter insertion. The co-administration of these drugs enables getting rid of patients' pain and fear at the time of catheter insertion and thereby increasing patient compliance. Furthermore, the addition of vasoconstrictor enables the enhancement of antimicrobial activity and analgesic effect and reduction of acting duration.

The local analgesics used include: for example, lidocaine hydrochloride, tetracaine hydrochloride, procaine hydrochloride, benzocaine hydrochloride, etidocaine hydrochloride, prilocaine hydrochloride, dibucaine hydrochloride, bupivacaine hydrochloride, cocaine hydrochloride, ethyl aminobenzoate, orihocainehydrochloride, oxethazainehydrochloride, mepivacaine hydrochloride, ropivacaine hydrochloride, and bupivacaine hydrochloride.

The vasoconstrictors used include: for example, α-adrenergics such as epinephrine, naphazoline, tetrahydrooine, oxymetazoline, xylometazolin, phenoxazoline, indanazoline, tramazoline and thimazoline.

Another effective apparatus embodying the invention is an assemblage for disinfecting the skin at a catheter or injection needle insertion site which includes 2 types of noninvasive components.

Referring to FIG. 2, there is shown a view of one example of antimicrobial administering assemblages constructed in accordance with the invention. The assemblage includes: a first component 200a and a second component 200b, as shown in the figure. The first component 200a includes a passive apparatus 201 for disinfecting the skin surface over a wide range. The second component 200b includes an active apparatus for disinfecting topically the skin surrounding the catheter or needle insertion site. The active apparatus includes: a donor electrode 200 containing an antimicrobial; a reference electrode 210 provided as a counter electrode of the donor electrode; and a power unit 220 for passing current between the donor electrode and the reference electrode. The first component 200a uses devices, such as patch and absorbent cotton both containing antimicrobial, alcohol or the like, as a pre-treatment agent. After the pre-treatment (e.g. wiping, applying a patch or absorbent cotton and stripping the same) with the first component 200a, an antimicrobial is administered with the second component 200b by, for example, iontophoresis.

Specifically, in the assemblage, the first component for disinfecting the skin surface over a wide range includes a passive apparatus containing at least one type of antimicrobial and the second component for disinfecting topically the skin surrounding the catheter or needle insertion site includes an active apparatus containing at least one type of active ingredient having antomicrobial activity. In the assemblage, the area on the skin to which the first component is applied and thereby resident microorganisms thereon can be eliminated (first area) is wider than the area on the skin to which the second component is applied and thereby resident microorganisms thereon can be eliminated (second area), the first area being 10 cm² or more, preferably 20 cm² or more, and more preferably 100 cm² or more and the second area being 1 to 100 cm², preferably 2 to 50 cm² or more.

The primary object of the first component is to apply an antimicrobial to the skin surface area in a short period of time and thereby kills the microorganisms on the skin surface over a wide range. Thus, the first component enables the prevention of the secondary infection caused by the microorganisms other than those on the skin surrounding the catheter insertion site. Preferably disinfection action is obtained from alcohols (e.g. ethanol and propanol), and preferably marked disinfection action is obtained by dissolving an antimicrobial in alcohols. The first component is excellent in convenience when applying a drug to the skin as well as in functions such as skin permeability to a drug and drug dosage control, since it is an alcohol-containing apparatus. In addition, the use of an alcohol solution makes the application of drug easy since alcohols volatilize in a short period of time. Furthermore, due to the short-time treatment, decrease in patient compliance can be kept to a minimum. Substances accelerating drug absorption may also be added to the alcohol solution.

The second component of the assemblage embodying the invention includes an active apparatus capable of delivering the antimicrobial having been applied to the skin surface with the first component to the depths of the skin and the appendages thereof reliably and in a short time. The active apparatus include, for example, apparatus including physical absorption accelerating means such as iontophoresis, electroporation, ultrasonics and heat, and iontophoresis is particularly effective means which provides outstanding drug delivering properties by electrical driving.

The types and the number of the antimicrobials used in the first and second components are not limited; however, when expecting potent effect on a specific microorganism, the active ingredient of the first component should be the same as that of the second component. Among the antimicrobials, chlorhexidine or the salts thereof exhibit potent disinfection action and is one of the effective drugs. On the other hand, when expecting potent effect on a wide variety of microorganisms, it is effective to add different active ingredients to the first and second components, respectively. Accordingly, in that case, one type or more of antimicrobials are appropriately selected and used.

The antimicrobials used in the apparatus as well as the assemblage of the invention are such that they lower the total activity of resident microorganisms on human skin, and preferably they are at least one selected from the group consisting of antivirals, antibacterials, chemotherapeutics, antibiotics, disinfectants and antimycotics; in particular, they include compounds having antimicrobial activity against at least one selected from the group consisting of Staphylococcus aureus, Staphylococcus epidermidis, coagulase-negative staphylococci, Micrococci, gram-positive bacilli, gram-negative bacillus acinetobacter, glucose-nonfermenting gram-negative rods, Candida, Serratia and methicillin-resistant bacteria.

### EXAMPLES

Examples of the invention and Comparative Examples will be described in further detail on the basis of experimental examples, hereinafter. However, it is to be understood that these examples are shown for illustrative purposes only and are not intended to limit the invention.

In Experimental Example 1, the influence of drug concentration on the activity of skin bacteria was examined. In Experimental Example 2, the influence of the amount of current applied on the activity of skin bacteria was examined, and in Experimental Example 3, the relation between the skin stimulation and the amount of current applied was examined. In Experimental Example 4, the influence of the antimicrobial administration methods on the activity of skin bacteria was examined. And in Experimental Example 5, the analgesic effect when co-administering an antimicrobial and a local analgesic was examined.

### Experimental Example 1

In Experimental Example 1, the influence of chlorhexidine glucuronate (Hibitane: manufactured by ZENECA Pharma) concentration on the activity of skin bacteria was examined adopting the passive administration and the iontophoretic administration to show the comparative results.

Hereinafter chlorhexidine glucuronate is referred to as “CHXD” and chlorhexidine as “CHX” for short. In the experiment, a horizontal diffusion cell (effective area; 1.77 cm²) was used and porcin skin incised with a dermatome (about 600 μm) was used as a diaphragm. Isotonic phosphate-buffered solution, pH 7.4, (containing 10 mM sodium chloride) was used as a receptor solution, and a silver electrode and a silver/silver chloride electrode were applied as an anode and a cathode, respectively. After the experiment, bacteria on the skin surface were taken by the medium contact method.

The stratum corneum was stripped from the porcin skin by the tape stripping method and bacteria on the skin surface were taken by the medium contact method at 1st, 5th, 10th, 15th and 20th stripping. After about 24 hr of incubation at 37° C., the number of colonies on each medium were determined visually and the total colony number on each medium was calculated (n=3, mean value±standard deviation).

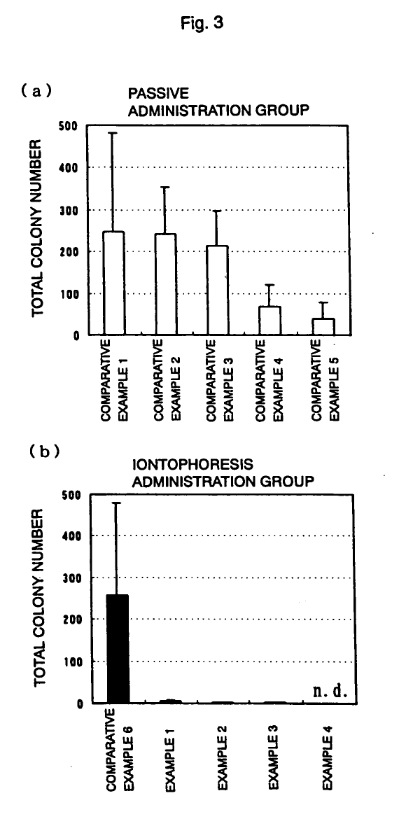

As shown in Table 1, in comparative examples 1 and 6, 0.09 w/v % sodium chloride was used in their respective donor solution, and in comparative examples 2 to 5 and examples 1 to 4, CHXD solutions having been prepared to give their respective concentrations (containing 0.09 w/v % sodium chloride) were used in the donor solutions. In comparative examples 1 to 5 passive administration was applied without energization, and in comparative example 6 and examples 1 to 4 DC iontophoresis (0.2 mA/cm²) was applied for 10 minutes. The results are shown in FIG. 3. FIG. 3(a) is a graph for a passive administration group and FIG. 3(b) for an iontophoretic administration group.

TABLE 1

| | | | Sodium |
|---|---|---|---|
| | | CHXD | chloride |
| Experimental | Administration | concentration | concentration |
| Example 1 | method | (w/v %) | (w/v %) |
| Comparative | Passive | 0 | 0.09 |
| Example 1 | | | |
| Comparative | Passive | 0.01 | 0.09 |
| Example 2 | | | |
| Comparative | Passive | 0.10 | 0.09 |
| Example 3 | | | |
| Comparative | Passive | 1.00 | 0.09 |
| Example 4 | | | |
| Comparative | Passive | 3.00 | 0.09 |
| Example 5 | | | |
| Comparative | Iontophoresis | 0 | 0.09 |
| Example 6 | | | |
| Example 1 | Iontophoresis | 0.01 | 0.09 |
| Example 2 | Iontophoresis | 0.10 | 0.09 |
| Example 3 | Iontophoresis | 1.00 | 0.09 |
| Example 4 | Iontophoresis | 3.00 | 0.09 |

As is apparent from FIG. 3, in Comparative Examples 1 and 6 in which no CHXD existed in the donor solution, the activity of residual microorganisms was high. In Comparative Examples 2 to 5 in which passive administration was applied, antimicrobial effect was observed at CHXD concentrations of 1.0 w/v % or more. However, when the passive administration was applied, the number of residual bacteria observed was still high and a large number of bacteria were observed particularly in the depths of stratum corneum. On the other hand, in examples 1 to 4 as a group in which the iontophoretic administration was applied, potent antimicrobial activity was exhibited at CHXD concentrations of 0.01 w/v % or more and almost all microorganisms were disappeared. It was confirmed that short term iontophoresis is highly effective in killing the resident bacteria in the stratum corneum of the skin even at low drug concentrations.

### Experimental Example 2

In Experimental Example 2, the influence of the amount of current applied on the activity of skin bacteria was assessed in the same in vitro test system as in experimental Example 1.

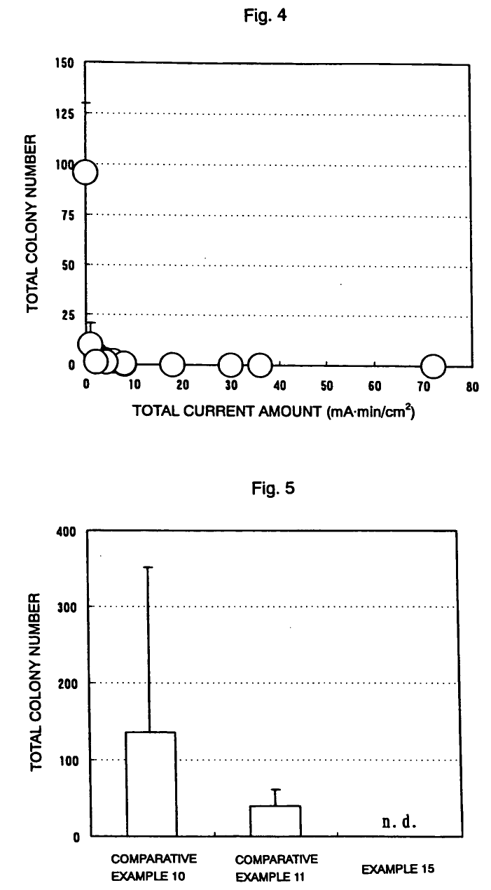

As shown in Table 2, in Comparative Example 7 as a control group the number of colonies on untreated skin was determined, and in Examples 5 to 14 and Comparative Examples 8 and 9 DC iontophoresis (1 w/v % CHXD, containing 0.09 w/v % sodium chloride) was applied under the respective conditions. All the results of Comparative Examples 7 to 9 and Examples 5 to 14 are plotted (mean value±standard deviation) in FIG. 4.

TABLE 2

| | | | Current |
|---|---|---|---|
| Experimental | Total current | Current | applying |
| Example 2 | amount (mA · min/cm²) | (mA/cm²) | duration (min) |
| Comparative | 0 | 0 | 0 |
| Example 7 | | | |
| Example 5 | 1.0 | 0.1 | 10 |
| Example 6 | 2.0 | 0.1 | 20 |
| Example 7 | 2.0 | 0.2 | 10 |
| Example 8 | 4.0 | 0.2 | 20 |
| Example 9 | 4.0 | 0.4 | 10 |
| Example 10 | 6.0 | 0.3 | 20 |
| Example 11 | 8.0 | 0.4 | 20 |
| Example 12 | 8.0 | 0.8 | 10 |
| Example 13 | 18.0 | 0.3 | 60 |
| Example 14 | 30.0 | 0.5 | 60 |
| Comparative | 36.0 | 0.3 | 120 |
| Example 8 | | | |
| Comparative | 72.0 | 0.3 | 240 |
| Example 9 | | | |

As is apparent from FIG. 4, it was confirmed that compared to Comparative Example 7 in which current was not applied, in Examples 5 to 14 and Comparative Examples 8 and 9, an iontophoretic administration group, in which the total amount of current applied is 1.0 mA·min/cm² or more, the number of bacteria in the stratum corneum of the skin was markedly decreased. Particularly in Examples 8 to 14, an iontophoretic administration group, in which the total amount of current applied is about 4.0 mA·min/cm² or more, the bacteria in the stratum corneum of the skin were almost completely disappeared.

### Experimental Example 3

In Experimental Example 3 the relation between the skin stimulation and the amount of current applied was examined.

Topical stimulation test on rabbit skin was carried out using Japanese White male rabbits (about 3.0 to 4.0 kg body weight) of which backs had been clipped with a pair of hair clippers, treated with a shaver and defatted and disinfected by rubbing with 70% ethanol aqueous solution-impregnated absorbent cotton. Commercially available donor electrode pads and reference gel (Trans QE: manufactured by IOMED) were applied to the rabbit back skin. The electrode pads were impregnated with 2 mL of donor solution. Comparative Examples 7 to 9 and Examples 5 to 14 were carried out under the same current application conditions as those of Experimental Example 2 shown in Table 2. After completing the application of current, the electrode pads were removed, and 24 hr after the removal, the skin stimulation to which the donor electrode had been applied was visually observed. The skin stimulation was assessed with the criteria grouped into the following 5 grades: no stimulation; very slight erythema; apparent erythema; moderate to severe erythema; and severe erythema to crusting. The results are shown in Table 3.

TABLE 3

| Experimental | Total current amount | |
|---|---|---|
| Example 3 | (mA · min/cm²) | Skin stimulation |
| Comparative | 0 | no change |
| Example 7 | | |
| Example 5 | 1.0 | no change |
| Example 6 | 2.0 | no change |
| Example 7 | 2.0 | no change |
| Example 8 | 4.0 | no change |
| Example 9 | 4.0 | no change |
| Example 10 | 6.0 | no change |
| Example 11 | 8.0 | no change |
| Example 12 | 8.0 | very slight erythema |
| Example 13 | 18.0 | very slight erythema |
| Example 14 | 30.0 | very slight erythema |
| Comparative | 36.0 | clear erythema |
| Example 8 | | |
| Comparative | 72.0 | clear erythema |
| Example 9 | | |

As shown in Table 3, it was observed that skin stimulation was dependent on the amount of current applied by iontophoresis.

### Experimental Example 4

In Experimental Example 4 the influence of the antimicrobial administration methods on the activity of skin bacteria was examined in the same in vitro test system as in Experimental Example 1.

Test conditions in each example are shown in Table 4. In Comparative Example 10, 70 w/v % ethanol solution containing 0.5 w/v % CHXD was applied to the skin for 3 min and in comparative example 11, 0.09 w/v % sodium chloride solution containing no drug was administered by DC iontophoresis (0.2 mA/cm², for 10 min) after the skin was treated with 70 w/v % ethanol solution containing 0.5 w/v % CHXD for 3 min. In Example 15, 0.01 w/v % CHXD solution (containing 0.09 w/v % sodium chloride) was administered by DC iontophoresis (0.2 mA/cm², for 10 min) after the skin was treated with 70 w/v % ethanol solution containing 0.5 w/v % CHXD for 3 min. The results are shown in FIG. 5.

TABLE 4

| Experimental | Administration | First step | Second step |
|---|---|---|---|
| Example 4 | method | administration | administration |
| Comparative | Passive | 0.5 w/v % CHXD | not applied |
| Example 10 | | 70 w/v % ethanol | |
| | | solution | |
| Comparative | passive/ | 0.5 w/v % CHXD | without drug |
| Example 11 | iontophoretic | 70 w/v % ethanol | |
| | 2 step topical | solution | |
| | administration | | |
| Example 15 | passive/ | 0.5 w/v % CHXD | 0.01 w/v % CHXD |
| | iontophoretic | 70 w/v % ethanol | |
| | 2 step topical | solution | |
| | administration | | |

As is apparent from FIG. 5, the passive treatment with CHXD-containing alcohol solution in Comparative Example 10 did not produce sufficient antimicrobial activity and in Comparative Example 11 in which current alone was applied after the passive treatment, activity of microorganisms remained in the stratum corneum of the skin. On the other hand, in the 2 step administration of Example 15 in which iontophoretic administration was carried out after the passive treatment, though the drug concentration was low, bacteria in the stratum corneum completely disappeared. The antimicrobial effect of the iontophoretic administration alone was significantly reinforced with the 2 step administration of Example 15.

### Experimental Example 5

In Experimental Example 5 the analgesic effect when co-administering an antimicrobial and a local analgesic was examined.

In the experiment, guinea pigs (Hartley strain, male) were used which had their backs clipped with a pair of hair clippers and shaved with an electric shaver and their skin surface wiped well with lukewarm water-impregnated gauze. The skin was stimulated with a stimulating needle on the right or left side relative to their back mesial line, and to the stimulation site at which the contraction reaction of the skin reliably showed up a donor electrode pad (TransQE: manufactured by IOMED) was applied and to other hair removed site a reference electrode was applied. The electrode pad was impregnated with 2 mL of donor solution and the solution of example 16 or 17 was administered by DC iontophoresis (0.2 mA/cm², for 10 min). In the 2 step topical administration method, 70 w/v % ethanol solution containing 0.5 w/v % CHXD, as the first step topical administering solution, was applied to the skin by the passive administration for 3 min. After this, the solution of Example 18 or 19, as the second step topical administering solution, was administered by DC iontophoresis (0.2 mA/cm², for 10 min). In comparative Example 12, untreated guinea pigs were used. After completing the application of current, the donor electrode application site was stimulated with a stimulating needle six times and the change with time in the contraction reaction of the skin was observed. The effectiveness of local anesthesia was assessed with the criteria shown in Table 5. The results are shown in Table 6.

### Example 16

| | |
|---|---|
| Chlorhexidine glucuronate | 1.00 w/v % |
| Sodium chloride | 0.09 w/v % |
| Purified water | suitable amount |

The above solution was administered by DC iontophoresis (0.2 mA/cm², for 10 min).

### Example 17

| | |
|---|---|
| Chlorhexidine glucuronate | 1.00 w/v % |
| Lidocaine hydrochloride | 1.00 w/v % |
| Epinephrine hydrochloride | 0.01 w/v % |
| Sodium chloride | 0.09 w/v % |
| Purified water | suitable amount |

The above solution was administered by DC iontophoresis (0.2 mA/cm², for 10 min).

### Example 18

| | |
|---|---|
| Chlorhexidine glucuronate | 1.00 w/v % |
| sodium chloride | 0.09 w/v % |
| Purified water | suitable amount |

After the treatment by the passive administration, the above solution was administered by DC iontophoresis (0.2 mA/cm², for 10 min).

### Example 19

| | |
|---|---|
| Chlorhexidine glucuronate | 1.00 w/v % |
| Lidocaine hydrochloride | 1.00 w/v % |
| Epinephrine hydrochloride | 0.01 w/v % |
| Sodium chloride | 0.09 w/v % |
| Purified water | suitable amount |

After the treatment by the passive administration, the above solution was administered by DC iontophoresis (0.2 mA/cm², for 10 min).

TABLE 5

| | Anesthesia score immediately |
|---|---|
| Number of reaction | after administration |
| React to 5th or 6th stimulation | − |
| React to 3rd or 4th stimulation | ± |
| React to 1st or 2nd stimulation | + |
| No reaction | ++ |

As shown in Table 6, it was confirmed that guinea pigs were analgesic to the pin pricking method in Examples 17 and 19 in which the administration solution contained lidocaine hydrochloride. On the other hand, in Examples 16 and 18 as well as Comparative Example 12 analgesic effect was not observed. When observing antimicrobial activity in Examples 16 to 19 by the medium contact method just like in Experimental Example 1, no bacteria were detected in any one of Examples.

TABLE 6

| Experimental Example 5 | Anesthesia score |
|---|---|
| Comparative Example 12 | − |
| Example 16 | − |
| Example 17 | ++ |
| Example 18 | − |
| Example 19 | ++ |

As described so far, the device for administering antimicrobial of the invention enables active ingredients to be transported directionally to the skin appendages in a reliable manner and in a short period of time by DC iontophoresis in which the total amount of current applied is, for example, 1 to 30 mA·min/cm² and realizes safe and efficient antimicrobial administration to living bodies. In addition, the assemblage of the invention, which is a combination of a passive apparatus and an active apparatus, enables antimicrobial activity to be significantly enhanced when an alcohol-containing apparatus is used as the passive apparatus and an iontophoresis apparatus as the active apparatus. The assemblage of the invention is clinically more effective and excellent in general-purpose properties as well as serviceability. Furthermore, the invention enables patients' pain at the time of catheter punctuation to be gotten rid of clinically by combining an antimicrobial with a local analgesic, which may increase patient compliance at the same time.

### INDUSTRIAL APPLICABILITY

According to the invention, an device for administering antimicrobial for killing resident microorganisms on human skin and thereby effectively preventing infections caused by the microorganisms and an assembly thereof can be obtained.

1. A device for administering antimicrobial using iontophoresis for disinfecting the skin at a catheter or needle insertion site, comprising:
(a) a donor electrode having an antimicrobial;
(b) a reference electrode provided as a counter electrode of the donor electrode; and
(c) a power unit for applying current between the donor electrode and the reference electrode in such a manner as to allow the total amount of current applied to be 1 to 30 mA·min/cm².

2. The device for administering antimicrobial according to claim 1, wherein the area on the skin to which the donor electrode is applied is 1 to 100 cm².

3. The device for administering antimicrobial according to claim 1, wherein the antimicrobial is at least one selected from the group consisting of antiviral, antibacterial, chemotherapeutic, antibiotic, disinfectant and antimycotic.

4. The device for administering antimicrobial according to claim 1, wherein the antimicrobial has antimicrobial activity against at least one selected from the group consisting of Staphylococcus aureus, Staphylococcus epidermidis, coagulase-negative staphylococci, Micrococci, gram-positive bacilli, gram-negative bacillus acinetobacter, glucose-nonfermenting gram-negative rods, Candida, Serratia and methicillin-resistant bacteria.

5. The device for administering antimicrobial according to claim 1, wherein the donor electrode is anodic and the antimicrobial contained therein may be chlorhexidine or a salt thereof.

6. The device for administering antimicrobial according to claim 1, wherein the donor electrode further comprises a local analgesic.

7. The device for administering antimicrobial according to claim 6, wherein the donor electrode is anodic and the local analgesic contained therein is lidocaine or a salt thereof.

8. The device for administering antimicrobial according to claim 1, characterized in that wherein the donor electrode further comprises a vasoconstrictor.

9. The device for administering antimicrobial according to claim 8, wherein the donor electrode is anodic and the vasoconstrictor contained therein is epinephrine or a salt thereof.

10. An assembly for administering antimicrobial for disinfecting the skin at a catheter or needle insertion site, comprising:
(a) a first component comprising a passive apparatus for disinfecting the skin surface over a wide range; and
(b) a second component comprising an active apparatus for disinfecting topically the skin surrounding the catheter or needle insertion site.

11. The assembly for administering antimicrobial according to claim 10, wherein each of the first and second components contains an antimicrobial.

12. The assembly for administering antimicrobial according to claim 10, wherein a first area as an area on the skin to which the first component is applied is wider than a second area as an area on the skin to which the second component is applied, the first area being 20 cm² or more and the second area being 1 to 100 cm².

13. The assembly for administering antimicrobial according to claim 10, wherein the first component contains an alcohol.

14. The assembly for administering antimicrobial according to claim 13, wherein the alcohols are alcohol is ethanol or isopropyl alcohol.

15. The assembly for administering antimicrobial according to claim 10, wherein the second component comprises:
(a) a donor electrode having an antimicrobial;
(b) a reference electrode provided as a counter electrode of the donor electrode; and
(c) a power unit for applying current between the donor electrode and the reference electrode in such a manner as to allow the total amount of current applied to be 1 to 30 mA·min/cm².

16. The assembly for administering antimicrobial according to claim 10, wherein the antimicrobial is at least one selected from the group consisting of antiviral, antibacterial, chemotherapeutic, antibiotic, disinfectant and antimycotic.

17. The assembly for administering antimicrobial according to claim 10, wherein the antimicrobial has antimicrobial activity against at least one selected from the group consisting of Staphylococcus aureus, Staphylococcus epidermidis, coagulase-negative staphylococci, Micrococci, gram-positive bacilli, gram-negative bacillus acinetobacter, glucose-nonfermenting gram-negative rods, Candida, Serratis and methicillin-resistant bacteria.

18. The assembly for administering antimicrobial according to claim 10, characterized in that wherein the first and the second components contain the same antimicrobial.

19. The assembly for administering antimicrobial according to claim 16, wherein the donor electrode is anodic and the antimicrobial is chlorhexidine or a salt thereof.

20. The assembly for administering antimicrobial according to claim 10, wherein the donor electrode further comprises a local analgesic.

21. The assembly for administering antimicrobial according to claim 20, wherein the donor electrode is anodic and the local analgesic is lidocaine or a salt thereof.

22. The assembly for administering antimicrobial according to claim 15, wherein the donor electrode further comprises a vasoconstrictor.

23. The assembly for administering antimicrobial according to claim 22, wherein the donor electrode is anodic and the vasoconstrictor is epinephrine or a salt thereof.
